(19) United States
(12) Patent Application Publication
(10) Pub. No.: US 2025/0161098 A1
(43) Pub. Date: May 22, 2025

(54) INFLATABLE OSTOMY DEVICE

(71) Applicant: BOSTON SCIENTIFIC SCIMED, INC., Maple Grove, MN (US)

(72) Inventors: Nathan Thomas CUMMINGS, Worcester, MA (US); Krista Lind HOLMSTROM, Savage, MN (US); Adrian MORA ELIZONDO, San Jose (CR); Amanda SALL, Marlborough, MA (US); Armando BARRANTES JIMENEZ, Heredia (CR); Damien Vincent NOLAN, Galway (IE); Oscar CASTRO, Grecia (CR)

(21) Appl. No.: 18/945,057

(22) Filed: Nov. 12, 2024

Related U.S. Application Data
(60) Provisional application No. 63/602,174, filed on Nov. 22, 2023.

(51) Int. Cl. A61F 5/449; A61F 2/94; A61F 5/44

(52) U.S. Cl. CPC A61F 5/449; A61F 2/94; A61F 5/4404; A61F 2220/0008

(57) ABSTRACT

An ostomy system includes a stent configured to be inserted through a stoma into a target organ coupled to the stoma. The stent extends from a proximal end to a distal end and includes a channel extending therethrough. The system also includes an adapter with a hub and a face rotatably coupled thereto. The hub is configured to be coupled to the distal end of the stent such that an opening extending therethrough is aligned and in communication with the channel of the stent. The face includes a first port and a second port. The face is rotatable relative to the hub between a first configuration in which the first port is aligned with and in communication with the opening of the hub and a second configuration in which the second port is aligned with an in communication with the opening of the hub.

### PRIORITY CLAIMS

The present disclosure claims priority to U.S. Provisional Patent Application Ser. No. 63/602,174 filed Nov. 22, 2023; the disclosure of which is incorporated herewith by reference

### FIELD

The present disclosure relates to ostomy devices and, in particular, relates to ostomy devices that facilitate collection and/or drainage of intestinal contents via a stoma.

### BACKGROUND

Ostomy procedures (e.g., colostomy, ileostomy) are performed for various patients with diseased or damaged bowels including, for example, colorectal cancer and diverticular disease. Current ostomy procedures include re-routing a part of the colon (or small intestine) to drain outside of the body by bringing an end of the colon to the abdominal wall, where edges of the colon are stitched to the skin to form an opening called a stoma. Stool is then able to drain from the stoma into a bag or pouch attached to the abdomen. Ostomy procedures, however, often result in post-operative complications and/or patient discomfort including, for example, leakage, odor, skin irritation from attachment of the ostomy bag, and social anxiety.

### SUMMARY

The present disclosure relates to an ostomy system. The system includes a stent configured to be inserted through a stoma into a target organ coupled to the stoma, the stent extending from a proximal end to a distal end and including a channel extending therethrough. The system also includes an adapter including a hub and a face rotatably coupled thereto, the hub configured to be coupled to the distal end of the stent such that an opening extending therethrough is aligned and in communication with the channel of the stent. The face includes a first port and a second port. The face is rotatable relative to the hub between a first configuration in which the first port is aligned with and in communication with the opening of the hub and a second configuration in which the second port is aligned with an in communication with the opening of the hub.

In an embodiment, the stent includes an anti-migration feature extending outward from an exterior surface thereof, the anti-migration feature being configured to engage tissue of an interior surface of the target organ to maintain a position of the stent relative to the stoma.

In an embodiment, the anti-migration feature is configured as a radially extending protrusion extending from the proximal end of the stent.

In an embodiment, the distal end of the stent includes a connector configured to be connected to the hub.

In an embodiment, the connector includes one of a snap-fit and a threading.

In an embodiment, the adapter further includes a strap configured to be received within a pair of holes extending through the hub so that the strap may be looped about a body of a patient to secure the adapter over the stoma.

In an embodiment, the each of the first and second ports is configured to be connected to a collection bag.

In addition, the present disclosure relates to an ostomy system. The system includes a stent configured to be inserted through a stoma into a target organ connected to the stoma, the stent extending from a proximal end to a distal end and including a channel extending therethrough. The system also includes an adapter including a hub configured to be connected to the distal end of the stent and a multi-port face rotatably coupled to the hub, the hub including an opening extending therethrough configured so that, in an operative configuration in which the hub is connected to the stent, the opening is aligned with and in communication with the channel of the stent, the face being rotatable relative to the hub between a first configuration in which a first port thereof is aligned with and in communication with the opening of the hub and the channel of the stent and a second configuration in which a second port of the face is aligned with and in communication with the opening of the hub and the channel of the stent.

The system further includes a first collection device configured to be connected to the first port so that, when the adapter is in the first configuration, material from passing from the target organ through the stoma is collected in the first collection device; and a second collection device configured to be connected to the second port so that, when the adapter is in the second configuration, material passing from the target organ through the stoma is collected in the second collection device.

In an embodiment, the stent includes a protrusion extending outward from an exterior surface thereof the protrusion being configured to engage tissue of the target organ to maintain a desired position of the stent relative to the stoma and the target organ.

In an embodiment, the protrusion extends radially outward from the proximal end of the stent.

In an embodiment, the one of the first and second collection devices includes a bag configured to receive material from the target organ therein, the bag extending longitudinally from a proximal end to a distal end, a length of the bag selected so that the bag is configured to be wrapped about a body of a user.

In an embodiment, the bag includes a loop slidable along a length thereof, the loop configured to hold first and second ends together to maintain the bag in a wrapped configuration.

In an embodiment, the bag includes a sensor configured to detect a fill level thereof.

In an embodiment, the bag includes a connector at the first end of the bag, the connector configured to releasably couple the bag to the one of the first and second ports.

In an embodiment, the bag includes a drain valve at the second end, the drain valve movable between a collecting configuration, in which contents are collectable within the bag, and a draining configuration, in which contents are drained from the bag thereby.

In addition, the present disclosure relates to a method for collecting waste passing from a target organ through a stoma. The method includes inserting a stent through the stoma such that the stent is received within an end of the target organ proximate the stoma; connecting a hub of an adapter to a distal end of the stent such that an opening of the hub is aligned with and in communication with a channel of the stent; and rotating a face of the adapter relative to the hub between a first position, in which a first port is aligned with and in communication with the opening of the hub and the channel of the stent so that a first collection bag connected to the stent collects material passing from the target organ through the stoma, and a second position, in which a second port of the face is aligned an in communication with the opening of the hub and the channel of the stent so that a second collection bag connected to the second port collects material passing from the target organ through the stoma.

In an embodiment, the method further includes moving the adapter from the first position to the second position when it is determined that the first collection bag is sufficiently full.

In an embodiment, a level of fill of the first collection bag is determined by a sensor attached thereto.

In an embodiment, the stent is inserted through the stoma such that the distal end thereof protrudes distally from the stoma to be connected to the hub external to the body.

In an embodiment, the stent is maintained in a desired position relative to the stoma via a protrusion extending outward from an exterior surface thereof to engage an interior surface of the target organ.

### BRIEF DESCRIPTION

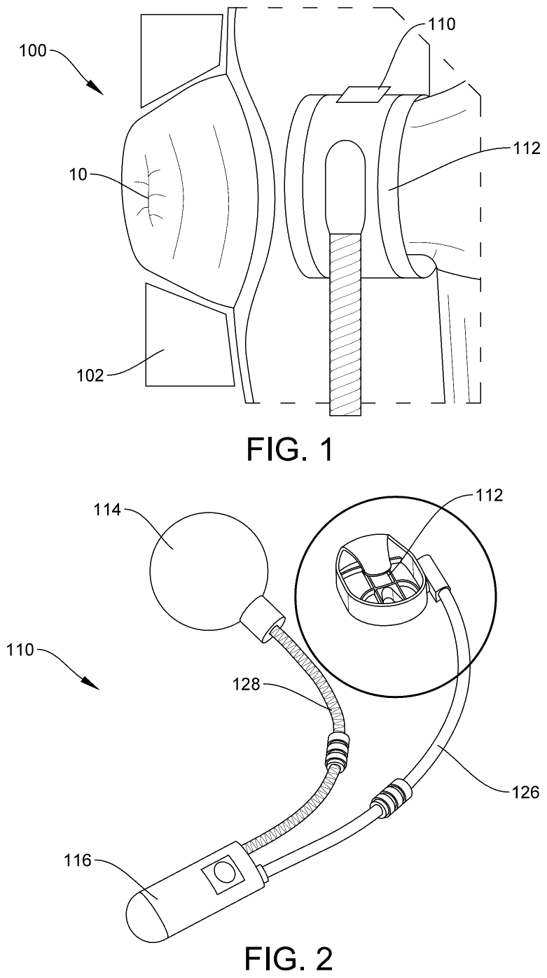

FIG. 1 shows a side view of an ostomy system according to an exemplary embodiment of the present disclosure;

FIG. 2 shows a perspective view of an artificial sphincter device according to the system of FIG. 1;

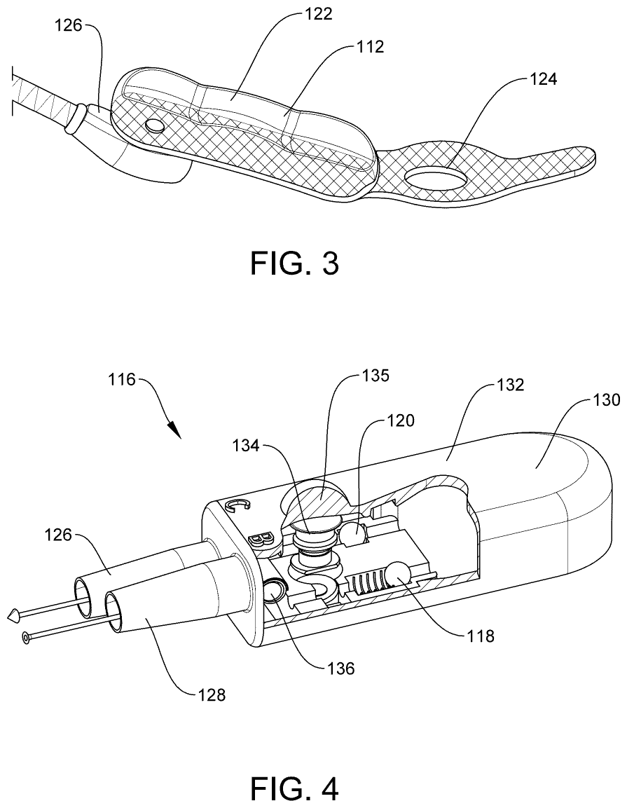

FIG. 3 shows a perspective view of a cuff portion of the artificial sphincter device according to FIG. 2;

FIG. 4 shows a partially transparent perspective view of a control pump assembly of the artificial sphincter device according to FIG. 2;

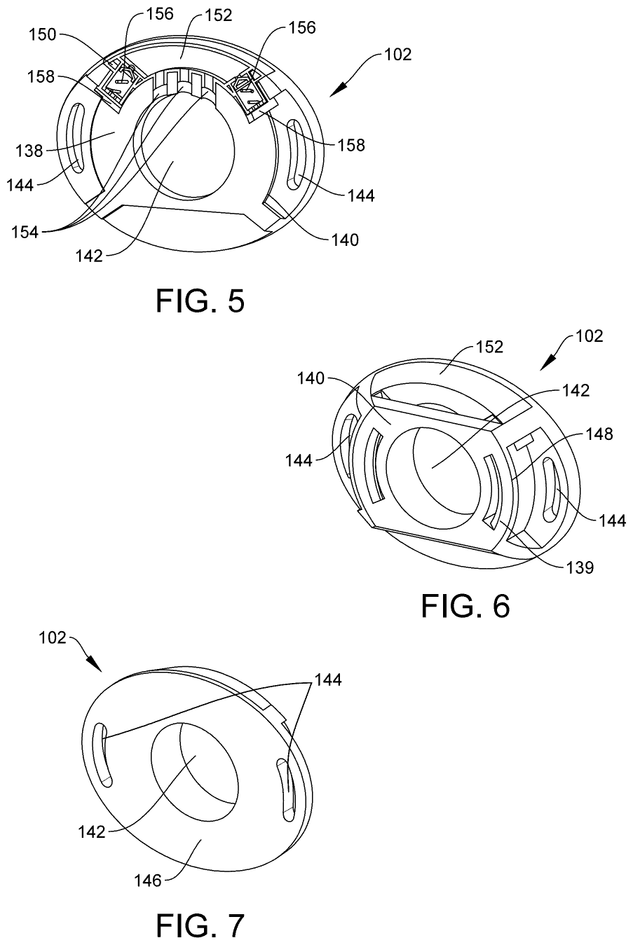

FIG. 5 shows a perspective view of an adapter according to the system of FIG. 1;

FIG. 6 shows another perspective view of the adapter according to the system of FIG. 1;

FIG. 7 shows yet another perspective view of the adapter according to the system of FIG. 1;

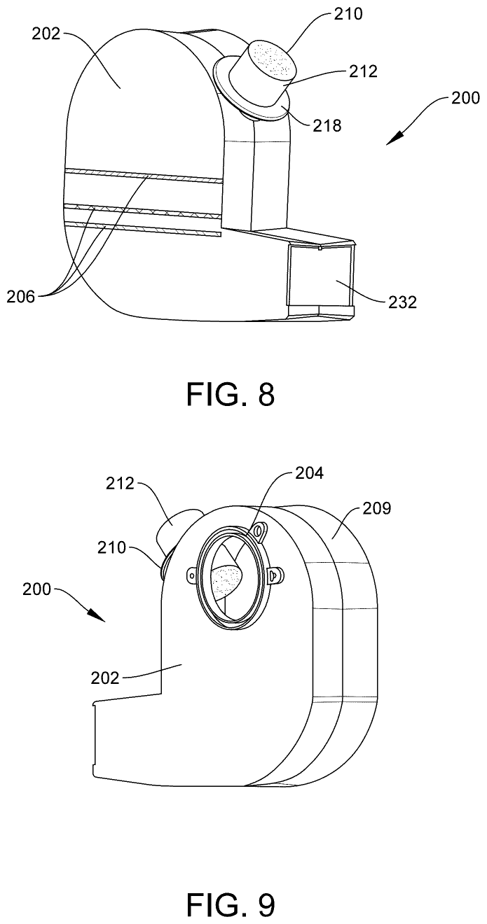

FIG. 8 shows a perspective view of an ostomy collection device according to an exemplary embodiment of the present disclosure;

FIG. 9 shows another perspective view of the ostomy collection device according to FIG. 8;

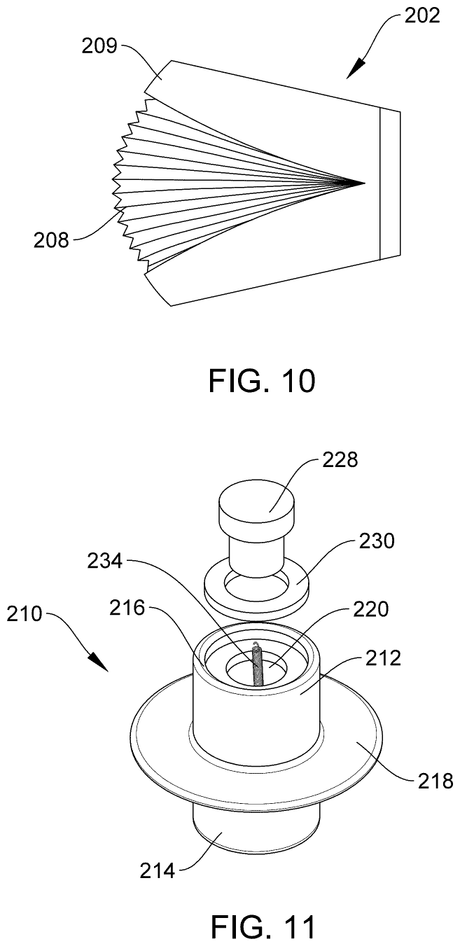

FIG. 10 shows a side view of an ostomy collection device according to another exemplary embodiment of the device of FIG. 8;

FIG. 11 shows an exploded perspective view of a pressure release valve according to the device of FIG. 8;

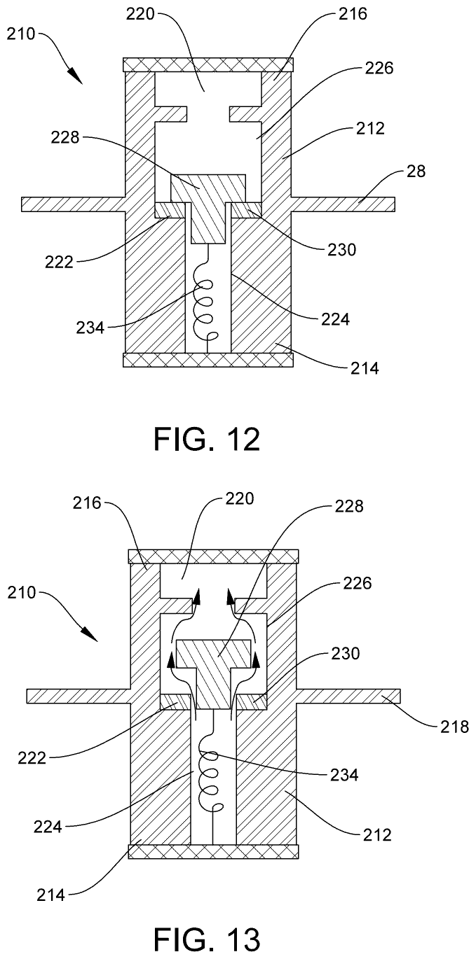

FIG. 12 shows a cross-sectional side view of the pressure release valve according to the device of FIG. 8, in a closed configuration;

FIG. 13 shows a cross-sectional side view of the pressure release valve according to the device of FIG. 8, in an open configuration;

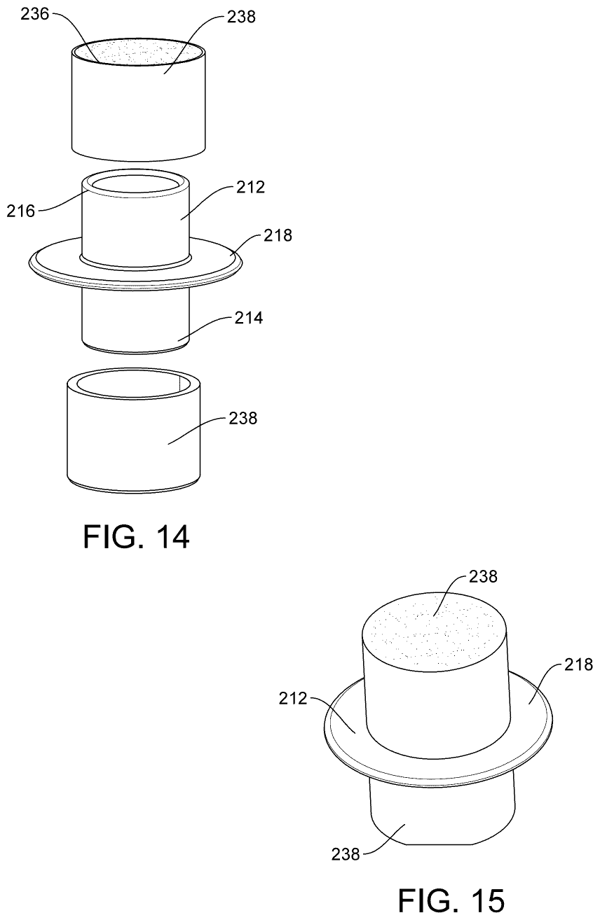

FIG. 14 shows an exploded perspective view of the pressure release valve and an odor-absorbing filter according to the device of FIG. 8;

FIG. 15 shows an assembled perspective view of the pressure release valve and the odor-absorbing filter according to the device of FIG. 8;

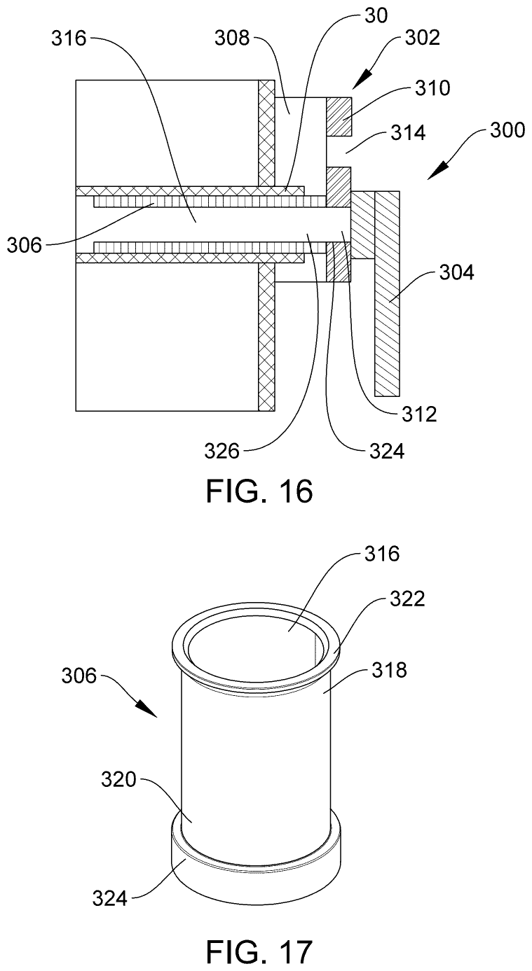

FIG. 16 shows a cross-sectional sideview of an ostomy system according to another exemplary embodiment of the present disclosure;

FIG. 17 shows a perspective view of a stent according to the exemplary system shown in FIG. 16;

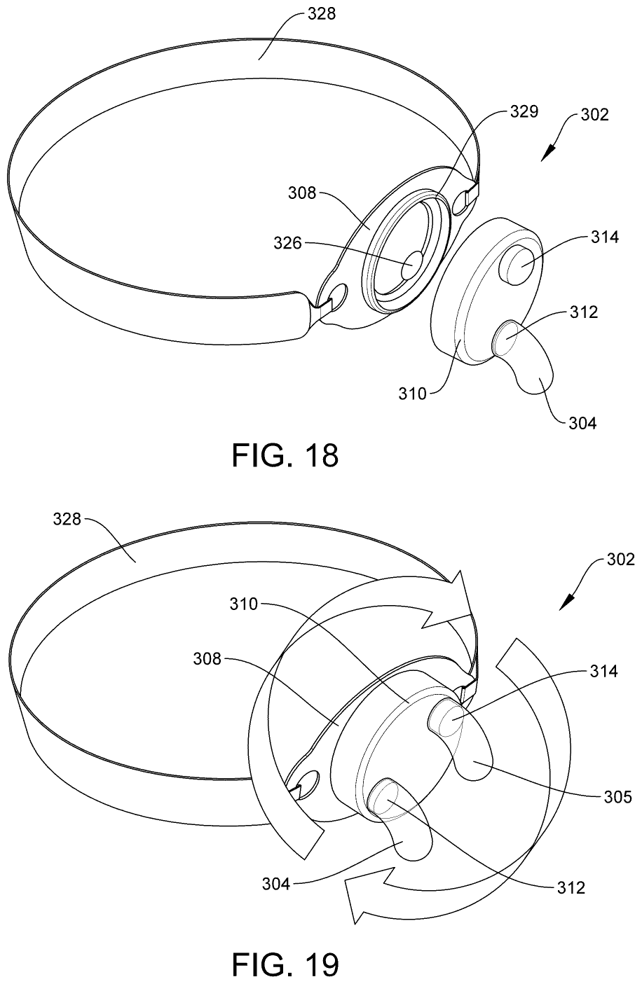

FIG. 18 shows a disassembled perspective view of an adapter according to the exemplary system of FIG. 16;

FIG. 19 shows an assembled perspective view of the adapter according to the exemplary system of FIG. 16;

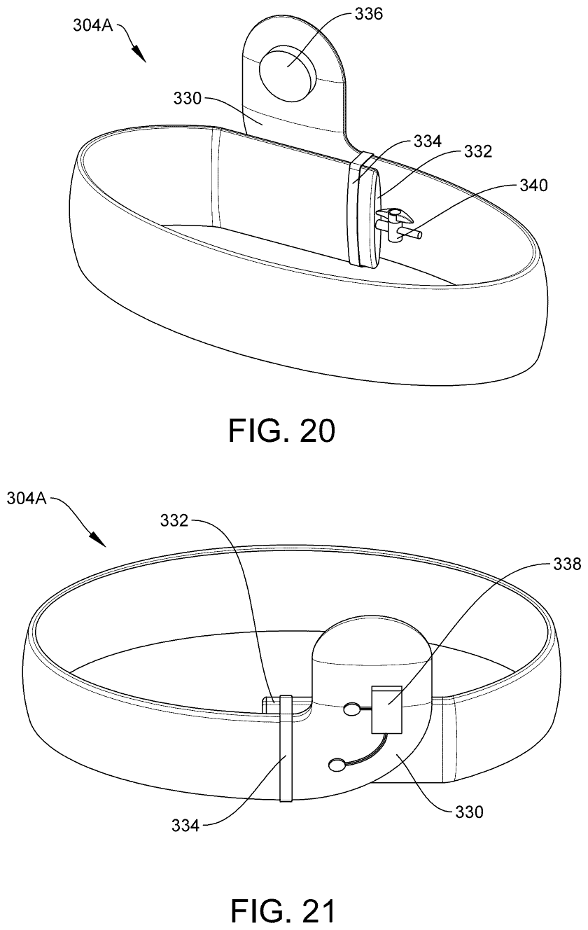

FIG. 20 shows a perspective view of a collection bag according to an exemplary embodiment of the present disclosure;

FIG. 21 shows another perspective view of the collection bag according to FIG. 21;

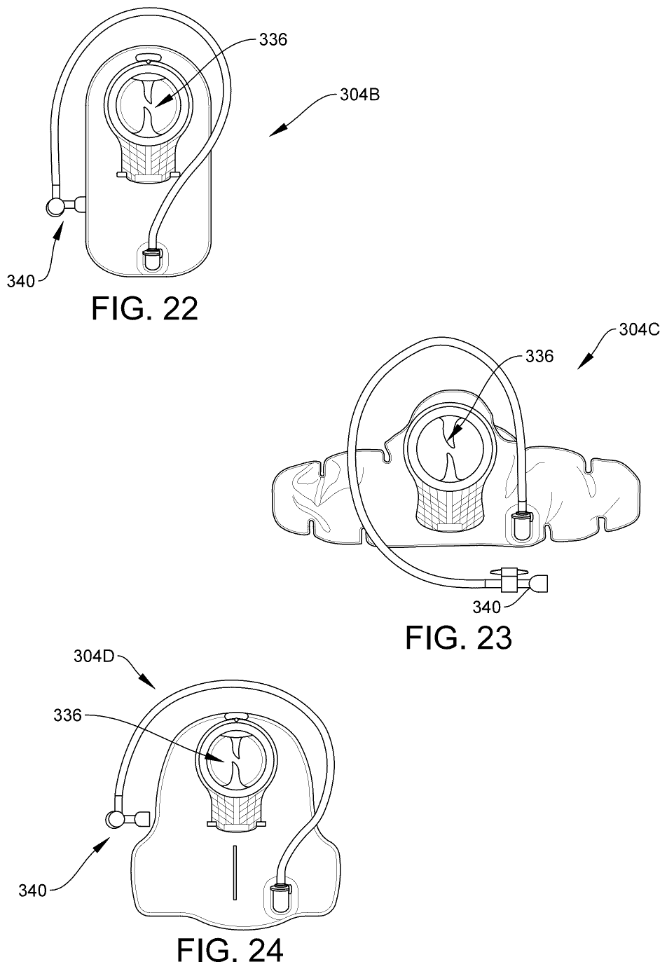

FIG. 22 shows a side view of a collection bag according to another exemplary embodiment;

FIG. 23 shows a side view of a collection bag according to yet another exemplary embodiment; and

FIG. 24 shows a side view of a collection bag according to another exemplary embodiment.

### DETAILED DESCRIPTION

The present disclosure may be further understood with reference to the following description and the appended drawings, wherein like elements are referred to with the same reference numerals. The present disclosure relates to an ostomy system and, in particular, relates to an ostomy device configured to facilitate a collection and/or drainage of intestinal contents via a stoma. Exemplary embodiments of the present disclosure comprise an external ostomy device configured to be attached to a stoma to facilitate collection and/or drainage of intestinal contents therefrom.

The exemplary ostomy device may include a variety of features such as a pressure release valve for preventing excessive pressure (over-pressure) from building up in a collection bag, a filter configured to filter odors, and/or features for indicating a fill level of the collection bag. Although the exemplary embodiments may specifically describe colostomy procedures in which an end of a portion of the colon is attached to an abdominal wall, it will be understood by those of skill in the art that the systems and methods of the present disclosure may be utilized for any ostomy procedure, including ileostomies, in which an end of a portion of the small intestine is re-routed to the abdominal wall. It will also be understood by those of skill in the art that although the exemplary embodiments may explicitly reference the colon, the exemplary ostomy system may be utilized for any portion of an intestine, including a bowel segment.

As shown in FIGS. 1-7, an ostomy system 100 according to an exemplary embodiment of the present disclosure comprises an adapter 102 configured to be positioned over a stoma 10 formed during an ostomy procedure. The adapter 102 is configured to facilitate attachment of a collection bag (not shown) thereto and may include any of a variety of features for improving patient comfort during collection of intestinal contents (e.g., stool, gas) within the collection bag. The adapter 102 may include, for example, a pressure-release valve for preventing over-pressure within the collection bag and a chamber configured to receive an odor absorbing filter.

In an exemplary embodiment, the adapter 102 is configured to be positioned over a traditionally formed stoma through which intestinal contents are permitted to flow freely. In another exemplary embodiment, as shown in FIG. 1, the adapter 102 is configured for use in conjunction with an artificial sphincter device 110 including an inflatable cuff 112 that has been, for example, subcutaneously implanted over an end of a portion of an intestine (e.g., colon, bowel), proximate the stoma 10.

The cuff 112 is configured to be moved between a deflated configuration, in which the stoma 10 is opened to allow intestinal contents to flow therethrough, through the adapter 102 and into the attached collection bag, and an inflated configuration, in which the stoma 10 is closed to prevent the flow of intestinal contents therethrough. In this embodiment, a user (e.g., ostomy patient) may wear the adapter 102 over the stoma 10 and attach a collection bag thereto when the user is ready to open the stoma 10 via the artificial sphincter device 110.

In an exemplary embodiment, as shown in FIG. 2, the artificial sphincter device 110 includes the cuff 112 along with a pressure regulating balloon 114 configured to regulate an amount of pressure applied to the colon (or bowel, or other portion of the intestine) during inflation of the cuff 112, and a control pump assembly 116 including an outflow valve 118 controlling a flow from the balloon 114 to the cuff 112 during inflation of the cuff 112 and an inflow valve 120 controlling a flow from the cuff 112 to the balloon 114 during deflation of the cuff 112.

The cuff 112, as shown in FIG. 3, includes a contoured shell 122 sized, shaped, and configured to be positioned, in an operative configuration, curved about the end of the colon, proximate the stoma 10. A length of the cuff 112 is selected so that the cuff 112 may be positioned to extend about and fixed in position relative to the colon so that the cuff 112 via, for example, a reinforced button hole 124. The contoured shell 122 is connected to the control pump assembly 116 via a first kink-resistant tubing 126 via which an inflation fluid (or gas) may be provided to and from to inflate and deflate the cuff 112, respectively, as desired.

The cuff 112 may be formed of any of a variety of biocompatible polymers configured to enlarge and/or expand when filled. For example, the cuff 112 may be formed of a silicone elastomer or any other suitable material as would be understood by those skilled in the art. In the deflated configuration, the cuff 112 does not apply significant pressure to the colon so that the stoma 10 is open to allow stool to pass therethrough from the intestine. In the inflated configuration, the cuff 112 applies radially inward pressure about the colon, closing the stoma 10 and preventing waste from flowing therethrough from the intestine.

In an exemplary embodiment, the balloon 114 is filled with an inflation fluid or gas such that an interior of the balloon 114 is at a positive pressure relative to the external environment. The balloon 114 may be available in multiple configurations to govern magnitude of internal pressure and, in some embodiments, may be operable so that a user may regulate an amount of pressure to be applied to the colon (or other segment of an intestine) via the cuff 112 to achieve a desired level of pressure. The balloon 114 is connected to the control pump assembly 116 via a second kink-resistant tubing 128 so that fluid is permitted to flow between the balloon 114 and the control pump assembly 116.

In an exemplary embodiment, as shown in FIG. 4, the control pump assembly 116 includes the outflow valve 118, the inflow valve 120, a deactivation valve poppet 134, and a fluid resistor 136 arranged within a housing 132. In an exemplary embodiment, each of the first and second valves 118, 120 may be configured as a check valve that is movable between an open configuration and a closed configuration to resist flow in a single direction. Fluid must flow through the deactivation valve poppet 134 to reach the balloon 114. When the user depresses the poppet 134 via, for example, a deactivation button 135 extending along the housing 132 and overlying the poppet 134, fluid flow is stopped until the poppet 134 is returned to an open configuration by squeezing a pump bulb 130 positioned along the housing 132 at, for example, an end thereof.

In an exemplary embodiment, during an initial activation, a pump bulb 130 along the housing 132 of the control pump assembly 116 is compressed so that the deactivation valve poppet 134 is moved from a closed configuration toward the open configuration. The inflow valve 120 seals to prevent ejection of a pump bulb volume to the cuff 112, while the outflow valve 118 opens to permit ejection of the pump bulb volume toward the balloon 114, facilitating flow from the cuff 112 via the pump bulb 130 to the pressure regulating balloon 114 during subsequent compressive cycles. As will be described in further detail below, the pump bulb 130 may be repeatedly compressed and released, as desired, until the cuff 112 is deflated and the stoma is open.

In particular, when a previously compressed pump bulb 130 is released, the inflow valve 120 opens, permitting flow from the cuff 112 into the pump bulb 130 to facilitate deflation of the cuff 112. When the user releases the pump bulb 130, it creates a vacuum, the outflow valve 118 resisting flow from the balloon 114 so that fluid is drawn from the cuff 112 through the inflow valve 120 into the pump bulb 130. The inflow valve 120 of this embodiment is biased toward the closed configuration during the compression of pump bulb 130 and is configured to be moved toward the open configuration when a previously compressed pump bulb 130 is released. The outflow valve 118 is in the closed configuration, flow is prevented from the cuff 112 into the pump bulb 130.

When the pump bulb 130 is pressed again, the outflow valve 118 is moved from the closed configuration toward the open configuration to allow fluid to flow from the pump bulb 130, through the second tubing 128, into the balloon 114. The pressure of the fluid within the housing 132 moves the inflow valve 120 from the open configuration toward the closed configuration to prevent fluid from passing therethrough, through the first tubing 126 and into the cuff 112. When the pump bulb 130 is released, the outflow valve 118 is once again moved toward the closed configuration, and the inflow valve 120 is moved toward the open position to allow flow therethrough and refill pump bulb 130 by drawing fluid from the cuff 112. Thus, the cuff 112 is deflated, permitting movement of intestinal contents (e.g., stool) through the stoma 10.

Accordingly, when it is desired to open the stoma 10, the user presses the pump bulb 130 to move the outflow valve 118 from the closed configuration toward the open configuration, ejecting fluid from the pump bulb 130 toward the balloon 114. Upon release of the pump bulb 130, the outflow valve 118 closes and the inflow valve 120 opens, permitting fluid to flow from the cuff 112, through the inflow valve 120 and into the housing 132. Upon compression of pump bulb 130, the hydrostatic pressure of the fluid within the housing 132 opens the outflow valve 118 so that the fluid may flow therethrough, back into the balloon 114. The pump bulb 130 compression/release cycle is repeated until the cuff 112 has been sufficiently deflated. When the cuff 112 has been deflated sufficiently to open the stoma 10, the pump bulb 130 is released and the outflow valve 118 reverts toward its closed configuration under its natural bias. Immediately upon cessation of pumping, the vacuum in the in the cuff 112 begins to draw fluid from the balloon 114, past the open deactivation valve poppet 134 and through the fluid resistor 136 back to the cuff 112. This continues until the pressure is equalized between the balloon 114, the cuff 112 and the pump bulb 130. The fluid resistor 136 ensures that the cuff 112 does not immediately refill (e.g., via a very slow fill rate), thus allowing time to evacuate intestinal contents through the stoma. If, however, it is desired to maintain the stoma 10 in an open configuration for an indefinite period of time, the deactivation button, which overlies the poppet 134, can be pressed following emptying of the cuff 112. In its closed configuration, the deactivation poppet 134 prevents fluid transfer from the balloon 114, thus preventing refill of the empty cuff 112.

In an exemplary embodiment, each of these components is configured to be internally implanted within, for example, a subcutaneous layer and/or intraabdominally, so that the user may control the artificial sphincter device by pressing and/or pinching the skin to press the pump bulb 130 and/or the deactivation button 134. It will be understood by those of skill in the art, however, that the control pump assembly 116 and/or the balloon 114 may be configured to be mounted external to the body of the user so that they are directly manipulable by the user. One or more components of the artificial sphincter device 110 may also be treated with antibiotics as would be understood by those skilled in the art. In an exemplary embodiment, for example, a material of the cuff 112 and/or the balloon 114 may be impregnated with antibiotics so that they may be slow-released over time to create a zone inhibiting the growth of infection-causing bacteria.

The adapter 102, as shown in FIGS. 5-7, is configured to be placed over a stoma 10 which has been formed in conjunction with the artificial sphincter device 110. In another embodiment, the adapter 102 is configured for placement over a traditionally formed stoma through which intestinal contents are permitted to freely flow. The adapter 102 of this embodiment includes a body 140 including an opening 142 extending therethrough, where the opening 142 sized, shaped, and configured to receive the stoma 10 therein. The body 140 of this embodiment is defined via a first surface 138 which, in an operative configuration, faces toward a skin of the user, and a second surface 139 which, in the operative configuration, faces away from the skin of the user. The opening 142 extends through the body 140 from the first surface 138 to the second surface 139.

In an exemplary embodiment, the body 140 is substantially ring shaped. It will be understood by those of skill in the art, however, that the body 140 of the adapter may have any of a variety of shapes and sizes so long as the body 140 is configured to be placed over the stoma 10. The body 140 may also include a connector 148 configured to attach the collection bag to the adapter 102. In an exemplary embodiment, the connector 148 includes a flange configured to engage a corresponding flange of the collection bag. It will be understood by those of skill in the art, however, that the connector 148 may have any of a variety of configurations so long as the connector 148 is configured to facilitate secure attachment and/or coupling of the collection bag thereto.

In an exemplary embodiment, the adapter 102 includes a pair of holes 144 along opposing sides thereof, with each of the holes configured to receive a portion of a belt therethrough so that the adapter 102 may be fixed in position over the stoma 10 via a belt or strap that is looped through the holes 144 and about the user's hips as would be understood by those skilled in the art. In addition to the belt and/or in place thereof, the adapter 102 may include an adhesive layer 146 over a first skin-facing surface 138 of the body 140. This adhesive layer 146 may be replaced as needed. It will be understood by those of skill in the art that the adapter 102, while replaceable if necessary, may be reused so that the adapter 102 remains over the stoma 10 through multiple changes of the collection bag.

The body 140 also includes a chamber 150 sized and shaped to receive an odor-absorbing (e.g., methane absorbing) filter 152 that may be replaced, as necessary. One or more channels 154 extend through the body 140 from the opening 142 to the chamber 150 so that, as intestinal contents are passed through the stoma 10, and thereby through the opening 142, odors may be absorbed via the filter 152.

The adapter 102 also includes one or more pressure release valves 156 fixed within the body, in communication with the chamber 150 and the filter 152 received therein. The pressure release valves 156 are movable between a closed configuration toward which the valves 156 are biased and an open configuration. In the closed configuration, each of the pressure release valves 156 is configured to block a corresponding one of the passages 158 extending through the body 140 between an interior of a collection bag attached to the adapter 102 and the chamber 150. In the open configuration, each valve 156 unblocks the corresponding passage 158 so that gas may be released from the collection bag therethrough to an exterior of the adapter 102. Since the passages 158 are in communication with the chamber 150, odor resulting from the release of gas is absorbed by the filter 152. The pressure release valves 156 are moved from the closed configuration toward the open configuration when a pressure within the collection bag has reached and/or exceeded a predetermined threshold pressure.

According to an exemplary method utilizing the system 100, a stoma 10 is created by attaching an end of a target organ (e.g., the colon or ileum) to an opening through an abdominal wall. In an exemplary embodiment, during creation of the stoma 10, the cuff 112 of the artificial sphincter device 110 is positioned about an end of a portion of portion of the intestine that has been passed through the opening (e.g., an incision) in an abdominal wall and stitched thereto to form the stoma 10 in any known manner. In an exemplary embodiment, the cuff 112 is implanted proximate the stoma 10, in a subcutaneous layer of the abdomen along with the control pump assembly 116, while the balloon 114 is positioned in the abdomen.

As described above, however, components of the artificial sphincter device 110 may be implanted in any of a variety of configurations-even externally-so long as the control pump assembly 116 is accessible by the user. As described above, the control pump assembly 116 is configured to move the cuff 112 between the deflated configuration and the inflated configuration to open and close the stoma 10, respectively. Although the exemplary method describes the use of the artificial sphincter device 110, as described above, the adapter 102 may also be utilized without the artificial sphincter device 110, over a traditionally formed stoma 10.

Upon creation of the stoma 10, the adapter 102 may be positioned thereover using, for example, a belt and/or an adhesive applied to the adapter 102. The adapter 102 is positioned over the stoma 10 so that the stoma 10 is received within the opening 142 extending through the body 140. A collection bag is attached to the adapter 102 via, for example, the connector 148. As intestinal waste flows from the intestine through the stoma 10 (e.g., when the artificial sphincter device is in the deflated configuration), the intestinal waste is received within the collection bag. Odors resulting therefrom are filtered via the odor absorbing filter within the chamber 150 of the adapter 102. The collection bag continues to collect the intestinal waste until it is full, at which point the collection bag may be detached from the adapter 102 while the adapter 102 remains positioned over the stoma 10. A new collection bag may then be attached to the adapter 102.

While the collection bag is collecting intestinal contents, the pressure release valve 156 releases gas from the collection bag when a pressure therein reaches and/or exceeds a predetermined threshold pressure. As described above, as gas is released, odors are absorbed via the filter 152. The filter 152 may be removed from the chamber 150 and replaced with a new filter, as necessary.

An ostomy collection device 200 according to an exemplary embodiment of the present disclosure, as shown in FIGS. 8-15, is configured for use in conjunction with any of the components of the system 100 described above and, in another embodiment, may be utilized alone. In an exemplary embodiment, as shown in FIGS. 8-9, the device 200 may include a bag 202 configured to be attached to a stoma via, for example, the adapter 102 described above with respect to the system 100. In another embodiment, the device 200 may be configured to be attached to the stoma via an alternate adapter such as, for example, a skin barrier or wafer. As will be understood by those of skill in the art, the skin barrier or wafer may include a connector such as, for example, a flange, configured to facilitate attachment of an ostomy bag such as the device 200, thereto. In an exemplary embodiment, the device 200 is equipped with a pressure release valve 210 for relieving pressure within the bag 202 and an odor-reducing filter 236.

In particular, in an exemplary embodiment, the bag 202 is fitted with a connector 204 corresponding to, for example, the connector 148 of the adapter 102 or a connector of an alternate adapter. In an exemplary embodiment, the connector 204 is configured to engage a corresponding flange of an adapter fitted about the stoma, to securely attach the bag 202 to the stoma. Once the bag 202 has been attached to the stoma, it is configured to collect intestinal contents passing through the stoma therein.

In an exemplary embodiment, the bag 202 includes a plurality of markings 206 thereon to indicate a level of fill of the bag 202. The markings 206 may, for example, indicate when the bag is half full, a third full, and/or a quarter full. A user may determine when to empty or drain the bag 202 based on the indicated level of fill—e.g., when the bag 202 is half full. In place of and/or in addition to the markings 206, the bag 202 may include pleating 208 along one or more edges 209 thereof, as shown in FIG. 10, to provide a sensory cue to the user that indicates the level of fill. In particular, the pleating 208 may be configured to expand as the bag 202 is filled. The bag 202 may also be available in a variety of colors corresponding to various skin tones.

The bag 202 may include a drainage port 232 through which intestinal contents may be drained from the bag 202 so that the bag 202 is not required to be changed as frequently. The drainage port 232 may be closed, until it is desired to drain the bag 202. In an exemplary embodiment, the drainage port 232 may be closed via, for example, a clip clipped thereover. It will be understood by those of skill in the art, however, that the drainage port 232 may be closed via any of a variety of closing elements including but not limited to, for example, a removable cap.

As described above, the device 200 includes the valve 210, configured to be attached to the bag 202, in communication with an interior thereof. The valve 210 is configured to automatically release gas received within the bag 202 when a pressure therein reaches and/or exceeds a predetermined threshold pressure. In an exemplary embodiment, as shown in FIGS. 11-13, the valve 210 includes a housing 212 extending from a first end 214 received within the bag 202 to a second end 216 extending to an exterior of the bag 202.

A channel 220 extends through the housing 212 from the first end 214 to the second end 216. The channel 220 includes a shoulder 222 therealong so that a first portion 224 of the channel 220 extending along a portion of the housing 212 that is received within the bag 202 has a cross-sectional area smaller than that of a second portion 226 of the channel 220 that extends along a portion of the housing 212 extending exterior to the bag 202. The housing 212 may also include a collar 218 extending radially outward from an exterior surface thereof, the collar 218 is configured to be adhered to a surface of the bag 202 to fix the housing 212 of the valve 210 relative to the bag 202.

The valve 210 further includes a plug which, in an exemplary embodiment is configured as a T-bar 228 movably housed within the housing 212 for movement between a closed configuration (e.g., shown in FIG. 12), in which the T-bar 228 is seated on the shoulder 222 to block flow through the channel 220, and an open configuration (e.g., shown in FIG. 13), in which the T-bar 228 is moved away from the shoulder—i.e., toward the second end 216 of the housing 212—to permit flow through the channel 220. A rubber O-ring 230 is, in this embodiment, positioned between the T-bar 228 and the shoulder 222 so that, when the T-bar 228 is in the closed configuration, the channel 220 is sealed. The T-bar 228 of this embodiment is biased toward the closed configuration via a biasing element 234 such as, for example, a spring. The biasing element 234 is configured to permit the T-bar 228 to be moved from the biased closed configuration toward the open configuration when subject to a predetermined threshold pressure. Thus, when the T-bar 228 is moved toward the open configuration, gas pressure build-up is released through the channel 220.

The device 200 further includes odor-reducing filters 236. In an exemplary embodiment, as shown in FIGS. 14-15, the odor-reducing filters 236 are configured as charcoal caps 238 that are configured to be capped over the first and/or second ends 214, 216 of the housing 212 although this is only one option and those skilled in the art will understand that any other suitable filters may be employed in like manner. The caps 238 are configured to be mounted over the first and/or second ends 214, 216 via a friction fit and/or may include an adhesive configured to facilitate attachment of the caps 238 to the housing 212. As will be understood by those of skill in the art, the caps 238 permit passage of gas therethrough while also providing odor-absorbing capabilities.

It will be understood by those of skill in the art that the described valve 210 automatically reduces pressure when a pressure within the bag 202 reaches and/or exceeds a predetermined threshold pressure and provides odorless degassing, reducing social discomfort.

As shown in FIGS. 16-21, an ostomy system 300 according to another exemplary embodiment of the present disclosure comprises, similarly to the system 100 described above, an adapter 302 configured to be positioned over a stoma 30 to facilitate drainage of intestinal contents (e.g., stool) therethrough. The adapter 302, however, is a multi-port adapter 302 configured to be coupled to multiple collection bags to facilitate easier collection bag exchange, prevent leakage, and reduce odor due to reduced exposure of the intestinal contents.

In an exemplary embodiment, as shown in FIG. 16, the adapter 302 is configured to be connected to the stoma 30 via a stent 306 inserted into the stoma 30. The adapter 302 includes a hub 308 configured to be coupled to the stent 306 and a face 310 rotatably coupled to the hub 308. The face 310 includes a first port 312 and a second port 314 configured so that the face 310 may be rotated relative to the hub 308 between a first configuration, in which the first port 312 is aligned with and in communication with a channel 316 of the stent 306 while the second port 314 is offset from the channel 316, and a second configuration, in which the second port 314 is aligned with and in communication with the channel 316 of the stent 306 while the first port 312 is offset from and closed to the channel 316.

Each of the first and second ports 312, 314 is configured to be connected to a collection bag. Thus, in the first configuration, intestinal contents may be collected within a first collection bag 304 connected to the first port 312. When the first collection bag 304 is full, the face 310 of the adapter 302 may be rotated relative to the hub 308 so that intestinal contents may be continued to be collected in a second collection bag 305 connected to the second port 314, reducing the risk of leakage and/or odor.

The stent 306 is sized, shaped, and configured to be inserted into a stoma 30. As shown in FIG. 17, the stent 306 extends from a proximal end 318 to a distal end 320, and includes the channel 316 extending therethrough. As will be understood by those of skill in the art, in an operative configuration, the proximal end 318 is configured to extend toward a user (e.g., ostomy patient) body while the distal end 320 extends away from the user body. In an exemplary embodiment, the stent 306 is configured to be inserted into the stoma 30 so that the proximal end 318 of the stent 306 is inserted into the stoma 30 and the distal end 320 protrudes slightly from the distal end of the stoma 30. The stent 306 may be formed of any of a variety of materials in any of a variety of configurations including for example, a partially or fully covered metal material, a rigid plastic material, or a flexible plastic material.

In an exemplary embodiment, the stent 306 includes an anti-migration feature 322 that includes any of a variety of structural features which, when the stent 306 is received in a desired position within the stoma 30, engage a portion of an intestine (e.g., colon) within which the stent 306 is received to reduce a likelihood of migration of the stent 306 within the intestine. The anti-migration feature 322 may include elements such as, for example, one or more flanges or other protrusion extending radially outward from an exterior surface of the stent 306 to engage tissue to lock the position of the stent 306 in place. In an exemplary embodiment, the anti-migration feature extends radially outward from the proximal end 318. It will be understood by those of skill in the art, however, that the anti-migration feature 322 may extend about and/or along any portion of the stent 306 so long as it is configured to engage an interior surface of the intestine in a manner designed to hold the stent 306 in a desired position.

The distal end 320 of the stent 306 includes a connector 324 configured to engage a portion of the adapter 302 as will be described in further detail below. The connector 324 may be configured as, for example, a structure facilitating a snap-fit or including threading configured to threadedly engage a correspondingly threaded portion of the adapter 302. It will be understood by those of skill in the art, however, that the connector 324 may include any of a variety of connection mechanisms so long as the connector 324 is configured to facilitate a connection between the stent 306 and the adapter 302.

As described above and as shown in FIGS. 18-19, the adapter 302 includes the hub 308 and the face 310 rotatably coupled thereto. In an exemplary embodiment, the hub 308 and the face 310 are configured to be rotatably coupled to one another via corresponding engaging structures 329 which facilitate rotation of the face 310 relative to the hub 308. In one example, the corresponding engaging structure 329 includes corresponding threading extending along portions thereof. In another example, the hub 308 may include a flange rotatably received within a correspondingly sized and shaped circumferential groove within a portion of the face 310.

In an exemplary embodiment, the hub 308 is configured to be attached to the connector 324 at the distal end 320 of the stent 306 and may include for, example, a corresponding snap-fit feature or threading configured to threadedly engage a corresponding threading on the connector 324. The hub 308 may include an opening 326 extending therethrough and is configured to be connected to the stent 306 so that, in an operative configuration, the opening 326 is aligned with and in communication with the channel 316 of the stent 306. In one exemplary embodiment, the opening 326 is offset relative to a central axis of the hub 308 and includes a connecting feature along an interior thereof (e.g., snap fit, threading) configured to engage the connector 324 of the stent 306.

In addition to the connecting features of the hub 308 that is configured to connect the hub 308 to the connector 324 of the stent 306, in an exemplary embodiment, the hub 308 may be held in position over the stoma 30 via a belt, strap, or band 328 that is looped about a body of the user (e.g., patient) and through holes extending through the hub 308, similarly to the adapter 102, described above with respect to the system 100. As would be understood by those skilled in the art, the band 328 aids in maintaining a position of the adapter 302 over the stoma 30 and may provide additional support for the first collection bag 304 and/or second collection bag 305 connected to the adapter 102 as it collects intestinal contents. As will be understood by those of skill in the art, the band 328 may be adjustable to accommodate users of all shapes and sizes.

As described above, the face 310 includes the first and second ports 312, 314 and is rotatably coupled to the hub 308 so that the face 310 may be rotated relative to the hub 308 between the first configuration, in which the first port 312 is aligned with and in communication with the stoma 30, and the second configuration, in which the second port 314 is aligned with and in communication with the stoma 30. Each of the first and second ports 312, 314 is configured to be connected to a collection bag. In an exemplary embodiment, the first port 312 is configured to be connected to the first collection bag 304 and the second port 314 is configured to be connected to the second collection bag 305.

It will be understood by those of skill in the art that the first and second collection bags 304, 305 may be sized, shaped, and configured to be similar to one another or, in another embodiment, may be sized, shaped, and configured to be different from one another. Each of the first and second collection bags 304, 305 may be connected to the corresponding one of the first and second ports 312, 314, respectively, via any of a number of connection mechanisms including, for example, a snap fit, a friction fit and/or a threaded connection as would be understood by those skilled in the art. It will be understood by those of skill in the art that although the face 310 of the adapter 302 is described and shown as including first and second ports 312, 314, that adapter 302 may include any number of ports so long as each of the ports of the adapter 302 is configured to be coupled to a collection bag to facilitate leak-proof and odor-proof changing between collection bags.

In the first configuration, the first port 312 is aligned with the opening 326 of the hub 308 so that the first port 312 is in communication with the channel 316 of the stent 306 while the second port 314 is out of alignment with the opening 326 and thus not in communication with the channel 316. Thus, in the first configuration, intestinal contents are permitted to flow from the stoma 30—through the channel 316 of the stent 306 received therein—through the opening 326 and the first port 312 to be collected in the first collection bag attached thereto.

When the first collection bag 304 is full and/or the user desires a new bag for collecting, the first collection bag 304 may be removed and the second collection bag 305 may be connected to the second port 314 of the face 310. In an exemplary embodiment, as shown in FIG. 18, one collection bag may be connected to the adapter 102 at a time, depending on the configuration of the adapter—e.g., the first configuration or the second configuration. In another embodiment, as shown in FIG. 19, multiple collection bags may be connected to the adapter 102 at the same time. For example, the second collection bag 305 may have previously been connected to the second port 314 so that upon filling of the first collection bag 304, the first collection bag 304 may be simply removed and the adapter 302 moved from the first configuration to the second configuration so that the user may proceed to collect intestinal contents in the second collection bag 305. In particular, the face 310 is rotated relative to the hub 308 to move the first port 312 out of alignment with the opening 326 so that the first port 312 is no longer in communication with the channel 316. Instead, the second port 314 is moved into alignment with the opening 326 so that the second port 314 is in communication with the channel 316 so that intestinal contents passing through the stoma 30 (through the channel) is collected within the second collection bag 305, which is connected to the second port 314.

In an exemplary embodiment, the face 310 is rotatably coupled to the hub 308 via a ratchet mechanism which permits rotation of the face 310 relative to the hub 308 in a single direction only. As would be understood by those skilled in the art, when the face 310 is coupled to the hub 308 via a threaded connection, such a ratchet mechanism would prevent the face 310 from being unthreaded from to the hub 308. In addition, such a ratchet mechanism would provide tactile feedback to the user to ensure that the face 310 is in a desired position relative to the hub 308 in each of the first and second configurations.

Each of the first and second collection bags 304, 305 may have any of a variety of configurations. In an exemplary embodiment, the first and second collection bags 304, 305 may be configured to be releasably coupled to each of the first and second ports 312, 314 and may formed of an elastomeric material which expands, as it is filled. The first and second collection bags 304, 305 may have any of a variety of shapes, sizes, and configurations, and may be the same as or different from one another. It will also be understood by those of skill in the art that although the system 300 is only shown and described with respect to two ports 312, 314 and two collection bags 304, 305, the system 300 may have any of number of ports and collection bags so long as the system 300 is configured to permit rotation of the adapter 302 to switch between collection bags.

In an exemplary embodiment, a collection bag 304A extends longitudinally from a first end 330 to a second end 332. A length of the collection bag 304A is preferably selected such that the collection bag 304A is configured to be looped about the user's body similarly to a belt. In particular, the first and second ends 330, 332 overlap one another and may be fixed about the user's body via, for example, a loop 334 configured to be slidable along a length of the collection bag 304A, similarly to a belt loop. The loop 334 is configured to hold the first and second ends 330, 332 together to maintain the collection bag 304A in a desired position about the body.

In an exemplary embodiment, the first end 330 includes a connector 336 configured to be connected to, for example, one of the first and the second ports 312, 314 of the adapter 302. The collection bag 304A may also include a sensor 338 which may indicate a fill level of the collection bag 304A. The sensor 338 may, in one embodiment, detect a conductivity of bag contents to sense a fill level of the bag 304A. In an embodiment, the sensor 338 is configured as an array of sensors configured to communicate with, for example, a user's smartphone or other user processing device via Bluetooth to communicate to the fill level to the user. The sensor 338 may be battery powered so that it does not require a wired power connection. Although the sensor 338 is shown as positioned at the first end 330 of the collection bag 304A, it will be understood by those of skill in the art that the sensor 338 may be located along any portion of the longitudinally extending collection bag 304A.

In an exemplary embodiment, the collection bag 304A further includes a drain valve 340 configured to facilitate drainage of intestinal contents collected within the collection bag 304A. When, for example, the sensor 338 indicates that the collection bag 304A has been filled to a point at which the collection bag 304A is considered full and/or requires drainage, the collection bag 304A may be drained via the drain valve 340. In an exemplary embodiment the drain valve 340 is positioned at the second end 332 of the collection bag 304A. It will be understood by those of skill in the art, however, that the drain valve 340 may be positioned along any portion of the collection bag 304A such that contents of the collection bag 304A may be drained therefrom.

Similarly to the device 200, the collection bag 304A may be available in a variety of colors configured to correspond to various skin tones. The collection bag 304A may also be available in a variety of lengths to accommodate user bodies of various shapes and sizes. In an exemplary embodiment, the collection bag 304A may be configured to be drained, washed, and reused.

Although the collection bag 304A is described as configured for use in conjunction with the adapter 302, it will be understood by those of skill in the art that the collection bag 304A may be attached to directly to the stoma 30. In another embodiment, the connector 336 may be configured to be attached to, for example, a flange of a skin barrier or wafer fitted about the stoma 30.

In addition, although the collection bag 304A is shown and described as having a longitudinally extending shape configured to be looped about a user body, it will be understood by those of skill in the art that the collection bag 304A may have any of a variety of shapes and sizes as shown via a collection bag 304B in FIG. 22, a collection bag 304C in FIG. 23, and a collection bag 304D in FIG. 24. The collection bags 304B, 304C, 304D may be otherwise similar to the collection bag 304A, including a connector 336 and a drain valve 340.

It will also be understood by those of skill in the art that the first and second collection bags 304, 305 may include any of the collection bags 304A, 304B, 304C, 304D. The first and second collection bags 304, 305 may have any of a variety of sizes, shapes, and configurations so long as they are configured to be connected to the first and second ports 312, 314, respectively.

According to an exemplary method of use of the system 300, upon formation of a stoma 30, the stent 306 is inserted through the stoma, into an end of the target organ (e.g., the colon) which forms the stoma 30. The stent 306 is inserted such that the proximal end 318 is inserted into the target organ and the distal end 320 extends just beyond a distal end of the stoma 30. As described above, the anti-migration feature 322 of the stent 306 reduces the risk of migration of the stent 306 relative to the stoma 30. The hub 308 of the adapter 302 is then connected to the connector 324 of the stent 306 so that the opening 326 of the hub 308 is aligned with and in communication with the channel 316 of the stent 306.

The face 310 of the adapter 302, which is rotatable relative to the hub 308, is first positioned in the first configuration relative to the hub 308 so that the first port 312 is in communication with the stoma 30 and the intestinal contents passing through the stoma 30 are collected within the first collection bag 304 connected to the first port 312. Once the first collection bag 304 is full and or requires changing, the adapter 302 is moved to the second configuration, in which the second port 314 is aligned with and in communication with the stoma 30 so that intestinal waste passing therethrough is collected in the second collection bag 305 connected to the second port 314.

It will be understood by those of skill in the art that each of the first and second collection bags 304, 305 may be removed from and/or connected to the first and second ports 312, 314 at any point during the above-described process. In some cases, in which the first and second collection bags 304, 305 include drain valves 340, the collection bags 304, 305 may remain connected to the first and second ports 312, 314 through multiple iterations of rotating the adapter 302 between the first and second configurations, so long as the first and second collection bags 304, 305 are emptied prior to the next phase of collection.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

1-15. (canceled)

16. An ostomy system, comprising:
a stent configured to be inserted through a stoma into a target organ coupled to the stoma, the stent extending from a proximal end to a distal end and including a channel extending therethrough; and
an adapter including a hub and a face rotatably coupled thereto, the hub configured to be coupled to the distal end of the stent such that an opening extending therethrough is aligned and in communication with the channel of the stent, the face including a first port and a second port, the face being rotatable relative to the hub between a first configuration in which the first port is aligned with and in communication with the opening of the hub and a second configuration in which the second port is aligned with an in communication with the opening of the hub.

17. The system of claim 16, wherein the stent includes an anti-migration feature extending outward from an exterior surface thereof, the anti-migration feature being configured to engage tissue of an interior surface of the target organ to maintain a position of the stent relative to the stoma.

18. The system of claim 17, wherein the anti-migration feature is configured as a radially extending protrusion extending from the proximal end of the stent.

19. The system of claim 16, wherein the distal end of the stent includes a connector configured to be connected to the hub.

20. The system of claim 19, wherein the connector includes one of a snap-fit and a threading.

21. The system of claim 16, wherein the adapter further includes a strap configured to be received within a pair of holes extending through the hub so that the strap may be looped about a body of a patient to secure the adapter over the stoma.

22. The system of claim 16, wherein each of the first and second ports is configured to be connected to a collection bag.

23. An ostomy system, comprising:
a stent configured to be inserted through a stoma into a target organ connected to the stoma, the stent extending from a proximal end to a distal end and including a channel extending therethrough;
an adapter including a hub configured to be connected to the distal end of the stent and a multi-port face rotatably coupled to the hub, the hub including an opening extending therethrough configured so that, in an operative configuration in which the hub is connected to the stent, the opening is aligned with and in communication with the channel of the stent, the face being rotatable relative to the hub between a first configuration in which a first port thereof is aligned with and in communication with the opening of the hub and the channel of the stent and a second configuration in which a second port of the face is aligned with and in communication with the opening of the hub and the channel of the stent;
a first collection device configured to be connected to the first port so that, when the adapter is in the first configuration, material from passing from the target organ through the stoma is collected in the first collection device; and
a second collection device configured to be connected to the second port so that, when the adapter is in the second configuration, material passing from the target organ through the stoma is collected in the second collection device.

24. The system of claim 23, wherein the stent includes a protrusion extending outward from an exterior surface thereof the protrusion being configured to engage tissue of the target organ to maintain a desired position of the stent relative to the stoma and the target organ.

25. The system of claim 24, wherein the protrusion extends radially outward from the proximal end of the stent.

26. The system of claim 23, wherein one of the first and second collection devices includes a bag configured to receive material from the target organ therein, the bag extending longitudinally from a proximal end to a distal end, a length of the bag selected so that the bag is configured to be wrapped about a body of a user.

27. The system of claim 26, wherein the bag includes a loop slidable along a length thereof, the loop configured to hold first and second ends together to maintain the bag in a wrapped configuration.

28. The system of claim 26, wherein the bag includes a sensor configured to detect a fill level thereof.

29. The system of claim 26, wherein the bag includes a connector at the first end of the bag, the connector configured to releasably couple the bag to the one of the first and second ports.

30. The system of claim 26, wherein the bag includes a drain valve at the second end, the drain valve movable between a collecting configuration, in which contents are collectable within the bag, and a draining configuration, in which contents are drained from the bag thereby.

31. A method for collecting waste passing from a target organ through a stoma, comprising:
inserting a stent through the stoma such that the stent is received within an end of the target organ proximate the stoma;
connecting a hub of an adapter to a distal end of the stent such that an opening of the hub is aligned with and in communication with a channel of the stent; and
rotating a face of the adapter relative to the hub between a first position, in which a first port is aligned with and in communication with the opening of the hub and the channel of the stent so that a first collection bag connected to the stent collects material passing from the target organ through the stoma, and a second position, in which a second port of the face is aligned an in communication with the opening of the hub and the channel of the stent so that a second collection bag connected to the second port collects material passing from the target organ through the stoma.

32. The method of claim 31, further comprising moving the adapter from the first position to the second position when it is determined that the first collection bag is sufficiently full.

33. The method of claim 32, wherein a level of fill of the first collection bag is determined by a sensor attached thereto.

34. The method of claim 31, wherein the stent is inserted through the stoma such that the distal end thereof protrudes distally from the stoma to be connected to the hub external to the body.

35. The method of claim 31, wherein the stent is maintained in a desired position relative to the stoma via a protrusion extending outward from an exterior surface thereof to engage an interior surface of the target organ.
